(19) United States
(12) Patent Application Publication
(10) Pub. No.: US 2018/0279931 A1
(43) Pub. Date: Oct. 4, 2018

(54) Dickerson Medical Meter

(71) Applicant: William Dickerson, Wichita, KS (US)

(72) Inventor: William Dickerson, Wichita, KS (US)

(21) Appl. No.: 15/474,843

(22) Filed: Mar. 30, 2017

(51) Int. Cl. A61B 5/145; A61B 5/0205

(52) U.S. Cl. CPC A61B 5/14532; A61B 5/024; A61B 5/02055

(57) ABSTRACT

The Dickerson medical meter assist diabetic people which manage their insulin levels and tells them the amount of insulin intake that they need at the time. The device uses a fingerprint sensor to read insulin levels and provides instructions if steps are needed to control blood sugar immediately. The Dickerson medical meter includes a USB port so that you can charger the internal battery and save readings for medical offices. The battery level indicator will let a user know when it is time for a recharge. The device can be used with liquid insulin. This device can also check your heart rate, temperature and oxygen levels.

### BACKGROUND OF INVENTION

### 1. Field of Invention

The invention relates to the field of diabetic monitoring and the intake of liquid insulin. The two inventions focuses on a meter for measuring blood sugar levels in the blood using a finger sensor, whereas the liquid insulin is the intake of a diabetic medicine that keep your blood sugar leveled.

### 2. Description of the Related Art

Diabetes is a fast-growing and destructive disease. Diabetes can lead to blindness, life threatening organ damage and heart disease. When you have been diagnosed as a diabetic you must monitor your blood sugar levels and by doing that you have to pluck your fingertip and place the blood on a script that's connected to the blood sugar machine, children often have a hard time cooperating with the needle situation to get their blood sugar but they do anyway. When a diabetic fail to take their insulin their blood sugar can get completely high or low and he or she could go into a life threatening coma.

Looking into the related art about diabetes you can see where there have been many new creations that could check your blood sugar other than the prick, but have all been unsuccessful.

### BRIEF SUMMARY OF INVENTION

The purpose of the Dickerson Medical Meter is for measuring blood sugar levels in the blood by using a finger sensor. The Dickerson medical meter shows the results on a screen that's visible and clear to read. The Dickerson medical meter also provides information on your pulse rate, temperature and oxygen levels, these components play a big role in everyone life but mainly in diabetics. The Dickerson medical meter may come in a variety of lighter colors, this item is small so it's easy to pick up and put in your pocket or travel bag. The Dickerson medical meter will be explained throughout this Patent.

The features of the invention which are believed to be novel are particularly pointed out and distinctly claimed in the concluding portion of the specification. These and other features, aspects and advantages of the present invention will become better understood with reference to the following drawings and detailed description.

### DESCRIPTION OF THE DRAWINGS

The figures accompany the written portion of this specification which illustrate embodiments and methods of use for the present invention, Dickerson Medical Meter, constructed and operative according to the teachings of the present invention.

FIG. 1 shows a perspective view illustrating the Dickerson medical meter showing a fingerprint sensor, the readout screen, the battery level indicator and the outer housing, providing the full color readout and a yellow color housing according to an embodiment of the present invention.

FIG. 2 shows a perspective view illustrating the Dickerson medical meter showing a close-up of the fingerprint sensor, and the battery level indicator in the yellow color housing according to an embodiment of the present invention.

FIG. 3 shows a perspective view illustrating a Dickerson medical meter showing a fingerprint sensor, a readout screen, a battery level indicator, and outer housing and a USB connector providing a full-color readout and grey color housing according to an embodiment of the present invention.

FIG. 4 shows a perspective view illustrating the Dickerson medical meter showing the fingerprint sensor, the readout screen, the battery level indicator, and the outer housing, providing the full color readout and a red color housing according to an embodiment of the present invention.

FIG. 5 shows a perspective view illustrating the Dickerson medical meter showing the USB connector providing the full readout, the battery level indicator, and a red color housing according to an embodiment of the present invention.

### DETAILED DESCRIPTION

As discussed above, embodiments of the present invention relate to a diabetic monitoring device and more particularly to a meter for measuring blood sugar levels in the blood using a finger sensor.

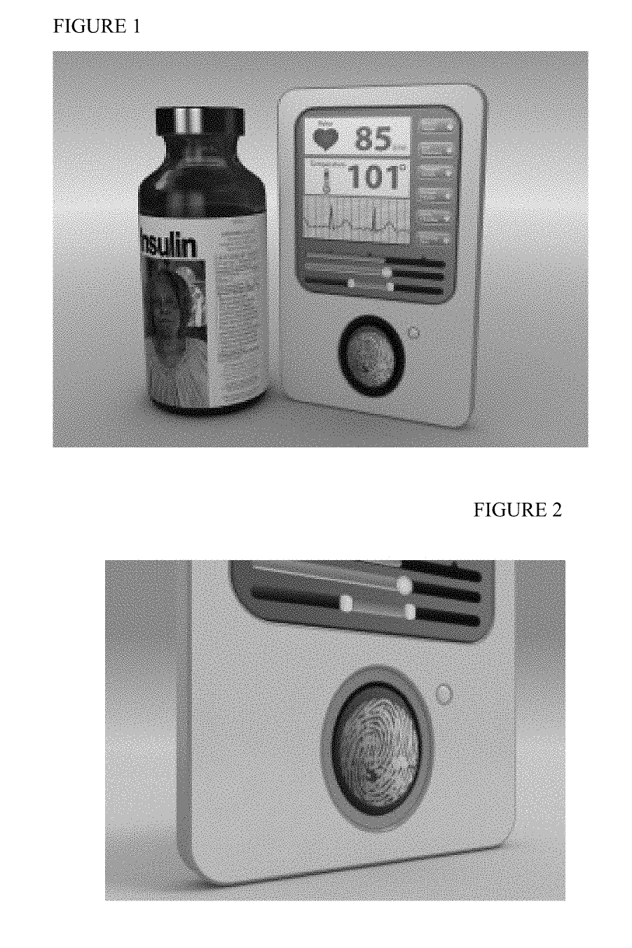

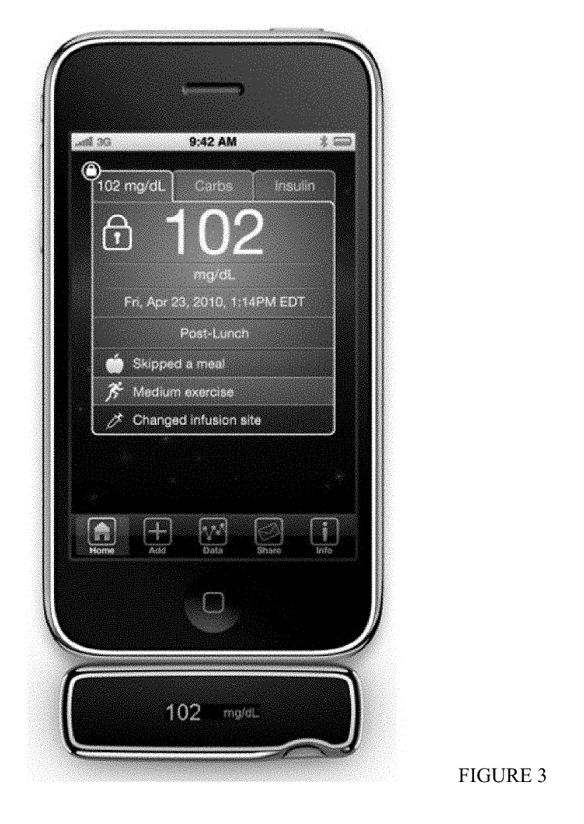

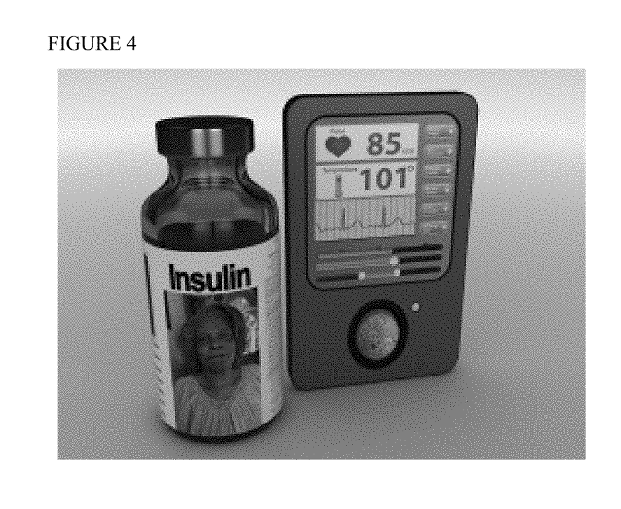

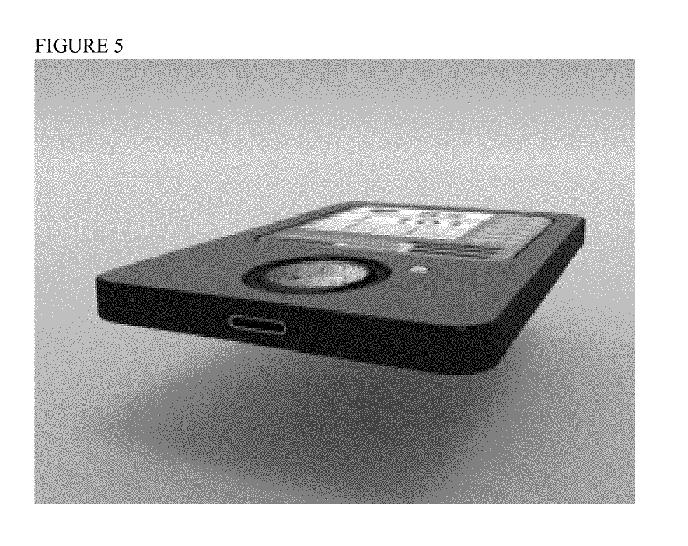

Referring to the drawings FIGS. 1-5, a Dickerson medical meter may relieve diabetics of the need to prick their fingers several times throughout the day. At regular intervals, they may be able to monitor their level of blood sugar and know how much insulin they need, by pressing on the sensor with one finger. The results of the reading and instructions for what they need to do all the time will be shown on its display screen. This monitoring meter has a USB port for connecting to a computer to submit results to a medical care provider, to maintain personnel records, and for recharging. There is an LED indicator light to warn users when their batteries are nearing depletion. The Dickerson medical meter may also be used by those on dialysis as well.

Diabetics will be able to regain greater control of their lives without being subjected to painful finger pricks and the need to carry bulky monitoring devices. This meter may also be used to indicate results of blood test and pregnancy tests. Parents will be able to test their child blood sugar without harming them in anyway.

What is claimed is new and desired to be protected by Letters Patent is set forth in the appended claim:

1. A Dickerson medical meter device is for measuring blood sugar levels in the blood using a finger sensor, you just place your finger on the sensor and it readout your blood sugar and heart rate. This information is described herein.
